(19) United States
(12) Patent Application Publication
(10) Pub. No.: US 2003/0119879 A1
(43) Pub. Date: Jun. 26, 2003

(54) Nicotine and chocolate compositions

(76) Inventors: Thomas Landh, Lund (SE); Nils-Olof Lindberg, Malmo (SE)

Correspondence Address: FULBRIGHT & JAWORSKI, LLP, 1301 MCKINNEY, SUITE 5100, HOUSTON, TX, 77010-3095, US

(21) Appl. No.: 10/270,945

(22) Filed: Oct. 15, 2002

Related U.S. Application Data
(60) Provisional application No. 60/329,571, filed on Oct. 15, 2001.

(51) Int. Cl. A61K 31/4439; A61K 9/68

(52) U.S. Cl. 514/343; 424/440

(57) ABSTRACT

The present invention is drawn to nicotine-containing pharmaceutical compositions that comprise chocolate and method of using the compositions in different therapies, such as nicotine replacement therapy.

This application claims priority to U.S. Provisional Application No. 60/329,571, which was filed on Oct. 15, 2001.

### BACKGROUND OF THE INVENTION

A. Field of Invention

This invention relates to novel pharmaceutical compositions of nicotine and use thereof. More particularly, the present invention relates to compositions comprising nicotine and chocolate, methods to prepare the compositions, and to methods for using the compositions in nicotine replacement therapy (NRT), including tobacco substitution and smoking cessation.

B. Related Art

Nicotine replacement therapy as a smoking cessation strategy has been successful in the past. Previous nicotine-containing compositions aiming towards the purpose of reducing nicotine craving for subjects wishing to stop their use of tobacco products include i.e., U.S. Pat. No. 3,845,217 disclosing chewable compositions, U.S. Pat. No. 4,579,858 disclosing high-viscous nicotine nose-drop compositions, U.S. Pat. No. 5,525,351 disclosing nicotine-containing saliva-soluble gels, U.S. Pat. No. 5,656,255 disclosing low-viscous nicotine-containing compositions suitable for nasal spray administration, U.S. Pat. No. 4,920,989 and U.S. Pat. No. 4,953,572 disclosing the use of inhalation aerosol, BP 1,528,391 and BP 2,030,862 disclosing liquid aerosol formulations adapted as mouth-sprays, and devices for transdermal delivery of nicotine.

A well-known side effect of nicotine is related to its concentration dependent local irritation. This adverse effect is particularly noticeable when nicotine formulations are applied topically, including the transmucosal administration (i.e., buccal and nasal) and transdermal administration routes.

UK Patent application GB 2230439A describes nicotine lozenges with a shell or coating containing an oral-acting local analgesic, preferably eugenol. Though not stated explicitly to be the cause of the so included local analgesic, the aforesaid disclosure is said to substantially ameliorate the sensation of burning in the mouth experienced with conventional nicotine lozenges. Similarly, nicotine-compositions formulated in lozenges containing local analgesic have been disclosed in AU 662877 in which the latter agent is said to temporarily interfere with taste receptors which is said to reduce the desire to eat.

The concentration of nicotine in several of the above-mentioned inventions, and product designs thereof, is hence limited by adverse effects caused by or related to its local irritation.

Prior art describes other capsules, tablets, and lozenges for oral delivery of nicotine. For example, WO 88/03803 discloses a chewable capsule filled with a liquid containing 0.1-10.0 mg of nicotine, together with additives for improving flavor and dispersion. The capsules are provided in a variety of pH values to allow the patient a choice of nicotine absorption rates, and are especially intended as an aid to quit smoking.

Another nicotine capsule formulation is disclosed by Jarvik et al. (Clinical Pharmacology and Therapeutics 1970;11:574) for ingestion as a smoking cessation aid. The subjects, according to the theory that intestinal absorption of nicotine could produce significant blood levels, however, apparently swallowed these capsules whole. The study showed a small but significant decrease in the number of cigarettes smoked by subjects, but no quantitative measurements of nicotine blood levels were obtained.

BE 899037 discloses a tablet containing 0.1 to 5 mg nicotine as a base or water-soluble acid salt as an aid for quitting smoking.

Shaw (for example in GB 2142822 and U.S. Pat. No. 4,806,356) describes a nicotine lozenge prepared from a mixture of inert filler material, a binder, and either pure nicotine or a nicotine-containing substance by cold compression.

U.S. Pat. No. 5,512,306 discloses a nicotine product for oral delivery in the form of an inclusion complex of nicotine and a cyclodextrin compound. It also discusses the use of various excipients and direct compression for manufacture of the product.

U.S. Pat. No. 5,662,920 discloses a nicotine lozenge that may contain candy taste flavorants, such as chocolate, orange, vanilla, as well as other flavorants. Their use also for taste-masking is though not suggested. Further, no amounts of these flavorants being sufficient for achieving a taste-masking effect is disclosed.

WO 97/42941 discloses a slowly erodible nicotine lozenge that allows delivery to the buccal mucosa over an extended period of time.

GB 2147501A discloses an oral dosage form comprising a microencapsulated active principle embedded in a soft sweet palatable matrix. This matrix may be chocolate. Nicotine is not suggested as an active principle.

The literature describes different designs of tablets for delivering nicotine to the mouth and to the digestive system.

Wesnes and Warburton (Psychopharmacology 1984; 82:147; Psychopharmacology 1986;89:55) discuss the use of nicotine-containing dextrose and magnesium hydroxide tablets. The subjects were instructed to keep the tablets in the mouth for some minutes before swallowing, in order to maximize contact with the buccal mucosa.

Several products based on the above mentioned patents are now marketed on an international scale. In addition, several nicotine lozenges are available as over-the-counter products in the UK Resolution lozenges, manufactured by Phoenix Pharmaceuticals and distributed by Ernest Jackson, which contain 0.5 mg nicotine together with the anti-oxidant vitamins A, C, and E. Stoppers lozenges, distributed by Charwell Pharmaceuticals Ltd., contain 0.5 mg nicotine and are available in chocolate, orange and peppermint flavors.

There are, however, subjects who may have cravings for higher doses of nicotine than those acceptable in applications of prior art and subjects that may not experience a decrease in other withdrawal symptoms because of unsatisfactory nicotine absorption. Furthermore, it has to date been difficult to deliver nicotine in a profile mimicking the nicotine blood levels achieved by consistent smoking, to satisfy cravings for nicotine in people who are attempting to quit smoking, and thus, to provide greater protection against relapse than nicotine replacement therapies is possible with hitherto known. Thus, absorption of nicotine in the use of currently marketed products and as disclosed in prior art of nicotine replacement therapies is not satisfactorily resembling the use of tobacco products, in particular smoking. With chewing gum, nicotine replacement therapy for smoking cessation blood peak levels of nicotine is reached after 30 minutes with venous blood nicotine levels about ⅓ to ⅔ of the levels attained when smoking (British Medical Journal 1976; 1:1043). A smoker will usually reach peak blood levels of nicotine 5-10 minutes after starting smoking. It is therefore desirable to provide improved compositions and methods which avoid the disadvantages of these conventional nicotine delivery devices and methods while providing an effective means for delivering nicotine for smoking cessation treatment, for reducing nicotine craving, and for treating other conditions responsive to nicotine therapy.

An attempt to solve the captioned problems is made with a nicotine-containing composition, preferably for buccal uptake, according to WO 00/30641. Herein is disclosed a composition comprising nicotine, at least one apolar component, at least one polar component and at least one surface-active component. Many apolar components are suggested, including lipids such as cocoa butter and cocoa butter alternatives, including cocoa butter equivalents (CBE), cocoa butter substitutes (CBS), cocoa butter replacers (CBR) and cocoa butter improvers (CBI). Anyhow, the composition according to WO 00/30641 has the disadvantage of insufficient taste-masking of nicotine and buffering agents, and the drawback of causing nausea with some users. WO 00/30641 does not disclose chocolate as an ingredient.

Chocolate has hardly been used as a vehicle for human pharmaceutical products, although chocolate-like pharmaceutical products of types laxatives exist. Chocolate-type veterinary products also exist. Ex-Lax®, being chocolated laxative pieces marketed by Novartis comprising sennosides, are formulated with a chocolate-like vehicle. In the 1950s, Purex marketed a laxative having phenolphtaleine that was formulated with chocolate. The Stoppers lozenges mentioned above do not comprise chocolate, but only chocolate flavors. Such chocolate flavors are not useful for the objectives of the present invention.

The present invention contemplates that a rapid buccal absorption of nicotine concomitantly with sufficient taste-masking of badly tasting ingredients, such as buffering agents and nicotine, is achieved through the use of nicotine-containing formulations comprising chocolate as a vehicle. No similar formulations have been disclosed hitherto. Thus, the present invention is the first to use chocolate as a vehicle for nicotine.

### BRIEF SUMMARY OF THE INVENTION

The present invention is directed to compositions for the therapeutic delivery of nicotine. The compositions comprising nicotine provide rapid transmucosal absorption of nicotine. More preferably, the compositions are used for therapeutic administration of nicotine. Yet further, the pharmaceutical compositions of nicotine are formulated for uptake buccally or by other mucosa in the oral cavity.

An embodiment of the present invention is a nicotine-containing pharmaceutical composition comprising chocolate as a vehicle. More specifically, the nicotine-containing pharmaceutical composition further comprises at least one buffering agent. The buffering agent is selected from the group consisting of sodium carbonates, sodium bicarbonates, sodium phosphates, sodium glycinates, sodium acetates, sodium gluconates, sodium glycerophosphates, potassium carbonates, potassium bicarbonates, potassium phosphates, potassium glycinates, potassium acetates, potassium gluconates, potassium glycerophosphates, ammonium carbonates, ammonium bicarbonates, ammonium phosphates, ammonium glycinates, ammonium acetates, ammonium gluconates, ammonium glycerophosphates and mixtures thereof. Yet further, the nicotine-containing pharmaceutical can also comprise at least one flavoring agent selected from the group consisting of mint, coffee, orange, vanilla and milk-butterscotch. Preferably, the composition is formulated for oral administration.

In specific embodiments, the nicotine-containing pharmaceutical composition is administered in a unit dose. The unit dose of the composition comprises from about 0.5 mg to about 10 mg of nicotine base, from about 5 mg to about 40 mg of a buffering agent and an effective amount of chocolate such that the amount of chocolate masks the nicotine taste. More preferably, the unit dose of the composition comprises from about 1 mg to about 6 mg nicotine base, about 95% (w/w) chocolate, and about 15 mg sodium carbonate.

Another embodiment of the present invention is a method for nicotine replacement therapy (NRT) comprising the step of administering to a subject in need of such therapy a unit dose of a nicotine-containing pharmaceutical composition, wherein the composition comprises nicotine, at least one buffering agent, and chocolate as a vehicle. Preferably, administering is via an oral route. Yet further, a second formulation of nicotine is also administered to the subject. The second formulation is administered via a device for transdermal administration of nicotine or is administered nasally or buccally or is administered via inhalation.

Yet further, another embodiment is a method of treating a subject suffering from nicotine addiction comprising administering to the subject the nicotine-containing composition of the present invention. Yet further, the composition may also be administered to a subject suffering from Alzheimer's disease, Parkinson's disease, Tourette's syndrome or ulcerative colitis. In further embodiments, the composition is also administered to a subject suffering from obesity. It is envisioned that the composition may also be used to control the weight of a subject.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description and is not intended as a definition of the limits of the present invention.

### DETAILED DESCRIPTION OF THE INVENTION

A. Definitions

As used herein, the use of the word “a” or “an” when used in conjunction with the term “comprising” in the sentences and/or the specification may mean “one,” but it is also consistent with the meaning of “one or more,” “at least one,” and “one or more than one.”

In the absence of explicit statements to the contrary, as used herein expressions like “comprising”, “including”, “having”, “with” and similar terminology shall not be understood to be exclusively restricted to the recited element(s), but shall be understood to allow for the presence of further elements as well, and shall be understood to cover any element(s) in integral, subdivided or aggregate forms, as well to imply the inclusion of a stated integer or step or group of integers or steps, but not the exclusion of any other integer or step or group of integers or steps.

The term “buccal” as used herein is defined as for uptake buccally or by other mucosa in the oral cavity.

The term “disintegration” as used herein denotes melting, solubilization, erosion or a combinatorial effect of these physical changes of the invention.

The term “oral administration” as used herein includes oral, buccal, enteral or intragastric administration.

The term “transmucosal administration” or “transmucosal delivery” as used herein means any system or device for the administration of a drug across a subject's mucosal membrane, including the oral mucosa, such as the buccal and sublingual mucosa, and other mucosal membranes, including rectal, nasal, and vaginal. See “Controlled Drug Delivery, Fundamentals and Applications”, 2nd Ed., Robinson and Lee, eds., Chapter 1, “Influence of Drug Properties and Routes of Drug Administration on the Design of Sustained and Controlled Release Systems”, Li et al., Marcel Dekker Inc.: New York, pp. 3-61 (1987).

The term “subject” as used herein, is taken to mean any mammalian subject to which a nicotine-containing composition is orally administered according to the methods described herein. In a specific embodiment, the methods of the present invention are employed to treat a human subject. Another embodiment includes treating a human subject in need of nicotine replacement therapy.

The term “taste-masking agent” as used herein refers to an agent that is added to a composition to mask the taste of a badly tasting component in the composition. For example, chocolate in the present invention masks the taste of nicotine.

The term “therapeutically effective amount” as used herein refers to an amount that results in an improvement or remediation of the symptoms of the disease or condition.

The term “treating” and “treatment” as used herein refers to administering to a subject an effective amount of a nicotine-containing composition so that the subject has an improvement in the disease or condition. The improvement is any improvement or remediation of the symptoms. The improvement is an observable or measurable improvement. Thus, one of skill in the art realizes that a treatment may improve the disease or condition, but may not be a complete cure for the disease or condition.

The term “prophylactic” as used herein is defined as a drug or agent, which acts to prevent a disease or condition, e.g., a vaccine.

B. Pharmaceutical Compositions

It is an object of the present invention to provide a nicotine-containing pharmaceutical composition. More specifically, it is the object of the invention to provide such a nicotine-containing composition for transmucosal, preferably buccal, delivery, which disintegrates and/or melts at body temperature with or without the aid of salivary fluid or mechanical erosion, or a combination thereof after which the formulation preferably shows adhesiveness towards the tissues in the oral cavity. This form of drug delivery provides for an efficient entry of active substances to the systemic circulation and reduces immediate metabolism by the liver and intestinal wall flora.

In preferred embodiments, the active ingredient of the composition is nicotine and chocolate is the vehicle for the active ingredient. Yet further, a buffering agent, for example sodium carbonate, can also be added to the composition.

The nicotine may be present in any suitable form, i.e., as free base, as a salt or as a complex. There is no need to use nicotine in a microencapsulated form. The free base is extremely volatile and is absorbed readily through mucous membranes and intact skin. The major problems reported for products based on nicotine free base originate from the volatility of the nicotine, its acrid, burning taste, the irritating sensation on the mucous membranes, and the decomposition of nicotine in the presence of oxygen. Previously, these problems have been alleviated, in part, through the use of nicotine's salt form, i.e., an acid addition salt or metal salt. The present invention utilizes chocolate as a vehicle to counter some of the problems associated with using a free base of nicotine, for example, burning taste. It is also envisioned that chocolate, in addition to acting as a taste-masking agent, may also serve as a smoothening and flavoring agent and/or as a filler and diluent agent. Thus, chocolate, as used herein masks the taste of nicotine and/or other badly tasting components, such as buffering agents.

Chocolate as used in the present invention is the vehicle or taste-masking agent. As used herein, the term vehicle and taste-masking agent are interchangeable. The chocolate that is used as the vehicle may be any form of chocolate, for example, but not limited to dark chocolate, milk chocolate or white chocolate. According to Industrial Chocolate Manufacture and Use, S. T. Beckett, ed., 2nd edition, Blackier Academic & Professional, London, 1994, p.382, chocolate is defined as a product obtained from cocoa nib, cocoa mass powder and sucrose with or without added cocoa butter, having a minimum dry cocoa solids content of 35%, at least 14% of dry non-fat cocoa solids and 18% cocoa butter. Chocolate has two major distinguishing characteristics: its flavor and its texture. A primary feature of the texture is that the chocolate must be solid at a temperature of 20-25° C. and yet melt rapidly in the mouth at 37° C. thereby being transferred to a liquid, which appears smooth to the tongue. The processing of chocolate is related to obtaining these two criteria. The higher the content of dry cocoa solids in the chocolate the better the taste masking effect of the chocolate in the present invention. Chocolate may also be defined according to different national directives, such as European Council Directive 2000/36/EC of Jun. 23, 2000, the old Council Directive 73/241/EEC of Jul. 24, 1973 (to be repealed from Aug. 3, 2003) and the US directive 21 CFR CH 1 (edition Apr. 1, 2000), part 163 Cacao products.

Other useful embodiments are obtained by exchanging some of the above-mentioned excipients for equivalently functioning alternative compounds. For example, the buffer sodium carbonate may be exchanged for e.g., carbonates, bicarbonates, phosphates, glycinates, acetates, gluconates or glycerophosphates of sodium, potassium or ammonium, or mixtures thereof. Most phosphates are thought less suitable because their taste usually is disagreeable and difficult to mask.

In specific embodiments, it is envisioned that the concentration ranges for the respective components of the formulation per unit dose are as follows: from about 0.5 mg to about 10 mg of nicotine (as base or salt, preferably hydrogen tartrate); about 5 mg to about 40 mg of a buffering agent; and a sufficient amount of chocolate. A sufficient or effective amount is an amount that masks the nicotine. For example, a sufficient amount of chocolate is the amount needed such that the subject does not taste the nicotine. Yet further, flavoring agents, such as mint, coffee, orange, vanilla and milk-butterscotch, may be added. The amount of flavoring agent is such a small amount that it does not interfere or decrease the amount of chocolate. In specific embodiments, the nicotine-containing pharmaceutical composition is administered in a unit dose. More preferably, the unit dose of the composition comprises from about 1 mg to about 6 mg nicotine base, about 95% (w/w) chocolate, and about 15 mg sodium carbonate.

The preferred formulation is a tablet melting in the mouth, weighing around 400 mg. Preferably, the tablet comprises nicotine (as base or hydrogen tartrate) sodium carbonate and dark chocolate. Another preferred embodiment is a tablet comprising nicotine (as base or hydrogen tartrate), sodium carbonate and white chocolate. Yet further, another composition may comprise nicotine (as base or hydrogen tartrate), sodium carbonate and milk chocolate. Still further, other oral drug dosage forms may also include, lozenges, capsules, or gum. The methods of manufacture of these formulations are known in the art, for example, as described in U.S. Pat. No. 4,806,356, which is incorporated herein by reference.

Upon formulation, solutions are administered in a manner compatible with the dosage formulation and in such amount as is therapeutically effective to result in an improvement or remediation of the symptoms. The formulations are easily administered in a variety of dosage forms such as ingestible tablets and the like. Some variation in dosage can occur depending on the condition of the subject being treated. The person responsible for administration can, in any event, determine the appropriate dose for the individual subject. Moreover, for human administration, preparations meet sterility, general safety and purity standards as required by FDA Office of Biologics standards.

C. Nicotine Therapy Replacement

It is the primary object of the present invention to provide a tobacco supplement or a tobacco substitute, for use in e.g. smoking cessation and nicotine replacement therapies, which provide the user with a satisfactory dose of nicotine so as to reduce tobacco withdrawal symptoms without causing unacceptable adverse effects. Yet further, it is envisioned that the addition of the taste-masking agent, chocolate, will reduce and/or eliminate the bad taste of the nicotine and/or other badly tasting components, such as buffering agents. Thus, the nicotine-containing composition of the present invention will be more desirable to the user.

A specific embodiment of the present invention is a method for nicotine replacement therapy comprising the step of administering to a subject in need of such therapy a unit dose of a nicotine-containing pharmaceutical composition, wherein the composition comprises nicotine, at least one buffering agent, and chocolate as a vehicle. Preferably, administering is via an oral route.

In further embodiments, the nicotine-containing composition may be administered in combination with a second formulation for nicotine replacement therapy. This second formulation may be a device for transdermal administration of nicotine, a spray for nasal, buccal or pulmonary uptake, a chewing gum, or a dosage form for oral or peroral use or any device for administration of tobacco.

D. Nicotine Therapy for Central Nervous System Disorders

Another aspect to the present invention is a method for the prevention and treatment of a central nervous system (CNS) disorder (i.e., Alzheimer's disease, Parkinson's disease, or Tourette's syndrome) by administering a nicotine-containing pharmaceutical composition to a subject susceptible to or suffering from such a disorder.

CNS disorders are a type of neurological disorder. CNS disorders can be drug induced; can be attributed to genetic predisposition, infection or trauma; or can be of unknown etiology. CNS disorders comprise neuropsychiatric disorders, neurological diseases and mental illnesses; and include neurodegenerative diseases, behavioral disorders, cognitive disorders and cognitive affective disorders. There are several CNS disorders whose clinical manifestations have been attributed to CNS dysfunction (i.e., disorders resulting from inappropriate levels of neurotransmitter release, inappropriate properties of neurotransmitter receptors, and/or inappropriate interaction between neurotransmitters and neurotransmitter receptors). Several CNS disorders can be attributed to a cholinergic deficiency, a dopaminergic deficiency, an adrenergic deficiency and/or a serotonergic deficiency. CNS disorders of relatively common occurrence include presenile dementia (early onset Alzheimer's disease), senile dementia (dementia of the Alzheimer's type), Parkinsonism including Parkinson's disease, Huntington's chorea, tardive dyskinesia, hyperkinesia, mania, attention deficit disorder, anxiety, dyslexia, schizophrenia and Tourette's syndrome.

It is known that nicotine has certain pharmacological effects, for example neurotransmitter release. Exemplary neurotransmitters that are released upon administration of nicotine include but are not limited to acetylcholine, dopamine (Rowell et al., J. Neurochem., Vol. 43, pp. 1593-1598 (1984); Rapier et al., J. Neurochem., Vol. 50, pp. 1123-1130 (1988); Sandor et al., Brain Res., Vol. 567, pp. 313-316 (1991)), norepinephrine (Hall et al., Biochem. Pharmacol., Vol. 21, pp. 1829-1838 (1972)), serotonin (Hery et al., Arch. Int. Pharmacodyn. Ther., Vol. 296, pp. 91-97 (1997)), and glutamate (Toth et al., Neurochem Res., Vol. 17, pp. 265-271 (1992)). Therefore, it is desirable to provide to a subject susceptible to or suffering from a CNS disorder a pharmaceutical composition containing nicotine, which elicits neurotransmitter release within the subject in order to prevent or treat a neurological disorder. In addition, the nicotine-containing composition of the present invention may also potentiate the pharmacological behavior of certain pharmaceutical compositions typically used for the treatment of certain CNS disorders. See, Sanberg et al., Pharmacol. Biochem. & Behavior, Vol. 46, pp. 303-307 (1993); Harsing et al., J. Neurochem., Vol. 59, pp. 48-54 (1993) and Hughes, Proceedings from Intl. Symp. Nic., S40 (1994). Thus, the nicotine-containing composition of the present invention can be used alone or in combination with other standard CNS therapies.

1. Alzheimer's Disease

Senile dementia of the Alzheimer's type (SDAT) is a debilitating neurodegenerative disease, mainly afflicting the elderly; characterized by a progressive intellectual and personality decline, as well as a loss of memory, perception, reasoning, orientation and judgment. One feature of the disease is an observed decline in the function of cholinergic systems, and specifically, a severe depletion of cholinergic neurons (i.e., neurons that release acetylcholine, which is believed to be a neurotransmitter involved in learning and memory mechanisms). See, Jones, et al., Intern. J. Neurosci., Vol. 50, p. 147 (1990); Perry, Br. Med. Bull., Vol. 42, p. 63 (1986) and Sitaram, et al., Science, Vol. 201, p. 274 (1978). It has been observed that nicotinic acetylcholine receptors, which bind nicotine and other nicotinic agonists with high affinity, are depleted during the progression of SDAT. See, Giacobini, J. Neurosci. Res., Vol. 27, p. 548 (1990); and Baron, Neurology, Vol. 36, p. 1490 (1986).

In certain embodiments, it is envisioned that administering the nicotine-containing composition of the present invention to a subject suffering form SDAT can ameliorate some symptoms of SDAT. It is contemplated that acute administration of the composition will activate nicotinic cholinergic receptors, and chronic administration of the composition will elicit an increase in the number of such receptors. See, Rowell, Adv. Behav. Biol., Vol. 31, p. 191 (1987); and Marks, J. Pharmacol. Exp. Ther., Vol. 226, p. 817 (1983).

2. Parkinson's Disease

Parkinson's disease (PD) is a debilitating neurodegenerative disease, presently of unknown etiology, characterized by tremors and muscular rigidity. A feature of the disease appears to involve the degenerative of dopaminergic neurons (i.e., which secrete dopamine). One symptom of the disease has been observed to be a concomitant loss of nicotinic receptors, which are, associated with such dopaminergic neurons, and which are believed to modulate the process of dopamine secretion. See, Rinne, et al., Brain Res., Vol. 54, pp. 167-170 (1991) and Clark, et al., Br. J. Pharm., Vol. 85, pp. 827-835 (1985).

In certain embodiments, it is envisioned that administering the nicotine-containing composition of the present invention to a subject suffering form PD may ameliorate symptoms of PD.

3. Tourette's Syndrome

Tourette's syndrome (TS) is an autosomal dominant neuropsychiatric disorder characterized by a range of neurological and behavioral symptoms. Typical symptoms include (i) the onset of the disorder before the age of 21 years, (ii) multiple motor and phonic tics although not necessarily concurrently, (iii) variance in the clinical phenomenology of the tics, and (iv) occurrence of quasi daily tics throughout a period of time exceeding a year. Motor tics generally include eye blinking, head jerking, shoulder shrugging and facial grimacing; while phonic or vocal tics include throat clearing, sniffling, yelping, tongue clicking and uttering words out of context. The pathophysiology of TS presently is unknown, however it is believed that neurotransmission dysfunction is implicated with the disorder. See, Calderon-Gonzalez et al., Intern. Pediat., Vol. 8(2), pp. 176-188 (1993) and Oxford Textbook of Medicine, Eds. Weatherall et al., Chapter 21.218 (1987).

A further embodiment of the present invention comprises administering to a subject suffering from TS the nicotine-containing composition of the present invention. It is envisioned that the nicotine-containing composition can be beneficial in suppressing the symptoms associated with TS. See, Devor et al., The Lancet, Vol. 8670, p. 1046 (1989); Jarvik, British J. of Addiction, Vol. 86, pp. 571-575 (1991); McConville et al., Am. J. Psychiatry, Vol. 148 (6), pp. 793-794 (1991); Newhouse et al., Brit. J. Addic., Vol. 86, pp. 521-526 (1991); McConville et al., Biol. Psychiatry, Vol. 31, pp. 832-840 (1992); and Sanberg et al., Proceedings from Intl. Symp. Nic., S39 (1994).

E. Treatment of Other Diseases or Disorders

Another aspect to the present invention is a method for the prevention and treatment of other diseases or disorders, such as ulcerative colitis or obesity by administering a nicotine-containing composition to a subject susceptible to or suffering from such a disorder.

Inflammatory bowel disorders or diseases (IBD) encompass a spectrum of overlapping clinical diseases that appear to lack a common etiology. IBD, however, are characterized by chronic inflammation at various sites in the gastrointestinal (GI) tract. Illustrative IBD are regional enteritis (or Crohn's disease), idiopathic ulcerative colitis, idiopathic proctocolitis, pouchitis and infectious colitis. Symptoms of IBD may include persistent diarrhea, abdominal pain, fever, weight loss, joint pain, skin lesions and general fatigue. The inflammatory conditions of ulcerative colitis are confined to the colon, unlike Crohn's disease, which can involve any portion of the intestinal tract.

Studies have suggested that an important epidemiolgic link exists between ulcerative colitis (UC) and a patient's smoking history. Several investigators have reported that the prevalence of UC in non-smokers is higher than in current smokers. Thus, a further embodiment of the present invention comprises administering to an individual suffering from UC the nicotine-containing composition of the present invention.

Yet further, it is envisioned that the nicotine-containing composition of the present invention can be used as a treatment for obesity or as a weight control therapy. It has been well established that smokers weight less than non-smokers. Intravenous nicotine infusion was shown to modestly increase the resting metabolic rate (6.5%) of smokers and non-smokers similarly. Also, in smokers and non-smokers alike, nasal nicotine solution insufflation significantly reduced the perceived taste intensity of dietary “fat”, but not “sweets”. From this, it appears that nicotine acts to decrease body weight through decreased calorie intake (i.e., appetite suppression) and increased metabolism. The mechanism for the observed appetite suppression is likely related to the increased serotonergic activity within the hypothalamus of the brain.

Thus, the present invention provides a therapeutic method to suppress appetite and/or prevent weight gain and/or induce weight loss in a subject in need of such therapy.

### F. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. Preparation of nicotine and chocolate composition

A tablet, weighing around 400 mg, having the following preferred composition (w/w): nicotine (1-6 mg as base or salt, preferably hydrogen tartrate); 15 mg sodium carbonate; and chocolate in a sufficient amount. The nicotine may also be present in a complex, e.g., with a cation exchange resin or with cyclodextrin.

The composition is prepared in the following way. Briefly, a part of the chocolate is melted. The solid components, i.e., nicotine, if in salt form, and sodium carbonate are added and mixed. A reduction of particle size of the solid components is performed by milling in a roll-refiner. If the solid components have already got the required particle size, e.g., by milling before the mixing with the chocolate, roll refining is dispensed with. After treatment, in the roll-re-finer, the mixture is mixed with the rest of the melted chocolate or remelted (if solidified) and mixed with the rest of the melted chocolate. Chocolate can be used as a raw material. Chocolate can also be produced in connection with the production of the embodiment. A mixing of the melt is performed in a suitable mixer. The liquid component, i.e., nicotine, if in liquid base form, is added. When chocolate is used as raw material a certain percentage of lecithin is already included (normally around 0.3%). Tablets or other solid dosage forms are subsequently made using suitable techniques, such as molding, extrusion or congealing, including pastillation, if necessary after suitable preconditioning. Also other suitable manufacturing methods may be used.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A nicotine-containing pharmaceutical composition comprising chocolate as a vehicle.

2. The nicotine-containing pharmaceutical composition of claim 1 further comprising at least one buffering agent, wherein the buffering agent is selected from the group consisting of sodium carbonates, sodium bicarbonates, sodium phosphates, sodium glycinates, sodium acetates, sodium gluconates, sodium glycerophosphates, potassium carbonates, potassium bicarbonates, potassium phosphates, potassium glycinates, potassium acetates, potassium gluconates, potassium glycerophosphates, ammonium carbonates, ammonium bicarbonates, ammonium phosphates, ammonium glycinates, ammonium acetates, ammonium gluconates, ammonium glycerophosphates and mixtures thereof.

3. The nicotine-containing pharmaceutical composition of claim 2, wherein the buffering agent is sodium carbonate.

4. The nicotine-containing pharmaceutical composition of claim 2 further comprising at least one flavoring agent selected from the group consisting of mint, coffee, orange, vanilla and milk-butterscotch.

5. The nicotine-containing pharmaceutical composition of claim 2, wherein a unit dose of the composition comprises from about 0.5 mg to about 10 mg of nicotine base, from about 5 mg to about 40 mg of the buffering agent and an effective amount of chocolate, wherein the amount of chocolate masks the nicotine taste.

6. The nicotine-containing pharmaceutical composition of claim 2, wherein a unit dose of the composition comprises from about 1 mg to about 6 mg nicotine base, about 95% (w/w) chocolate, and about 15 mg sodium carbonate.

7. The nicotine-containing pharmaceutical composition of claim 2, wherein the composition is formulated for oral administration.

8. A method for nicotine replacement therapy comprising the step of administering to a subject in need of such therapy a unit dose of a nicotine-containing pharmaceutical composition, wherein the composition comprises nicotine, at least one buffering agent and chocolate as a vehicle.

9. The method of claim 8, wherein the buffering agent is selected from the group consisting of sodium carbonates, sodium bicarbonates, sodium phosphates, sodium glycinates, sodium acetates, sodium gluconates, sodium glycerophosphates, potassium carbonates, potassium bicarbonates, potassium phosphates, potassium glycinates, potassium acetates, potassium gluconates, potassium glycerophosphates, ammonium carbonates, ammonium bicarbonates, ammonium phosphates, ammonium glycinates, ammonium acetates, ammonium gluconates, ammonium glycerophosphates and mixtures thereof.

10. The method of claim 9, wherein the buffering agent is sodium carbonate.

11. The method of claim 8, wherein the unit dose comprises from about 0.5 mg to about 10 mg of nicotine base, from about 5 mg to about 40 mg of the buffering agent and an effective amount of chocolate, wherein the effective amount of chocolate masks the nicotine taste.

12. The method of claim 8, wherein the unit dose comprises from about 1 mg to about 6 mg nicotine base, about 95% (w/w) chocolate, and about 15 mg sodium carbonate.

13. The method of claim 8, wherein administering is via an oral route.

14. The method of claim 8 further comprising administering to said subject a second formulation of nicotine.

15. The method of claim 14, wherein the second formulation is administered via a device for transdermal administration of nicotine.

16. The method of claim 14, wherein the second formulation is administered nasally or buccally.

17. The method of claim 14, wherein the second formulation is administered via inhalation.

18. A method of treating a subject suffering from nicotine addiction comprising administering to said subject the composition of claim 5.

19. A method of treating a subject suffering from Alzheimer's disease comprising administering to said subject the composition of claim 5.

20. A method of treating a subject suffering from Parkinson's disease comprising administering to said subject the composition of claim 5.

21. A method of treating a subject suffering from Tourette's syndrome comprising administering to said subject the composition of claim 5.

22. A method of treating a subject suffering from ulcerative colitis comprising administering to said subject the composition of claim 5.

23. A method of treating a subject suffering from obesity comprising administering to said subject the composition of claim 5.

24. A method of controlling the weight of a subject comprising administering to said subject the composition of claim 5.
